(19) United States
(12) Patent Application Publication
(10) Pub. No.: US 2013/0017155 A1
(43) Pub. Date: Jan. 17, 2013

(54) DUAL CT/MRI NANOPARTICLE CONTRAST AGENT

(71) Applicants: Kevin Bennett, Chandler, AZ (US); Simon Lubek, Tempe, AZ (US); Maria Veronia Clavijo-Jordan, Tempe, AZ (US)

(72) Inventors: Kevin Bennett, Chandler, AZ (US); Simon Lubek, Tempe, AZ (US); Maria Veronia Clavijo-Jordan, Tempe, AZ (US)

(21) Appl. No.: 13/637,678

(22) PCT Filed: Mar. 31, 2011
(86) PCT No.: PCT/US11/30682

§ 371 Date: Sep. 27, 2012

Related U.S. Application Data
(60) Provisional application No. 61/320,102, filed on Apr. 1, 2010.

(51) Int. Cl. C07K 14/435; B32B 1/00; A61K 49/14; A61K 49/04; B82Y 30/00

(52) U.S. Cl. 424/9.32; 530/400; 424/9.42; 428/402; 977/773

(57) ABSTRACT

The invention relates to a new tungsten-iron-Ferritin nanoparticle and uses thereof in imaging.

### RELATED APPLICATIONS

The present application claims the benefit of priority of U.S. Provisional Application No. 61/320,102, which was filed Apr. 1, 2010. The entire text of the aforementioned application is incorporated herein by reference.

### FIELD OF THE INVENTION

The present invention relates to a new imaging contrast agent and methods of use of the same.

### BACKGROUND OF THE INVENTION

Non-invasive imaging systems have become an essential part of modern medicine for obtaining the necessary information to diagnose various diseases. Important imaging techniques include, for example, Positron Emission Tomography (PET), Single Photon Emission Computed Tomography (SPECT), Magnetic Resonance Imaging (MRI) and Computed Tomography (CT). Each of these techniques relies on the use of contrast agents that allow imaging of tissues in vivo.

MRI and CT techniques are not dependent on tissue depth, and do not require radioisotopes and are used as diagnostic techniques that allows non-invasive imaging of optically opaque subjects and provides contrast among soft tissues at high spatial resolution. Gadolinium and magnetite nanoparticles have been used as contrast-enhancing agents for MRI.

In the majority of clinical applications, the MRI signal is derived from protons of the water molecules present in the materials being imaged. The image intensity of tissues is determined by a number of factors. The physical properties of a specific tissue, such as the proton density, spin lattice relaxation time (T1), and the spin-spin relaxation time (T2) often determine the amount of signal available. Depending on the properties of the contrast agents, the T1 (longitudinal) or T2 (transverse) weighted images or both may be altered. Methods to increase the resolution of MRI imaging include: extending the scan time, using high efficiency coils, increasing field strength, and increasing the accumulation of contrast agent in cells or tissue.

A number of compositions termed “contrast agents” have been developed to provide enhanced contrast between different tissues. Contrast agents commonly affect T1, T2 or both. In general, contrast agents are made potent by incorporating metals with unpaired d or f electrons. For example, T1 contrast agents often include a lanthanide metal ion, usually Gd³⁺, that is chelated to a low molecular-weight molecule in order to limit toxicity. T2-agents often consist of small particles of magnetite (FeO—Fe₂O₃) that are coated with dextran. Both types of agents interact with mobile water in tissue to produce contrast; the details of this microscopic interaction differ depending on the agent type.

MRI contrast agents have been tested in imaging of the liver, spleen, gastrointestinal tract and their cancers, detection of other cancers, and cardiovascular disease. When administered systemically, nanoparticles typically accumulate in the liver, spleen, and bone marrow, all of which are dependent on the reticulo endothelial system (RES). Furthermore, prior contrast agents have generally labeled healthy cells rather than malignant cells, making it difficult to identify small tumors and metastases. This “filtering” of nanoparticles has generally limited their use for imaging to the specific tissues in which they accumulate. For example, Endorem™ and AMI25™, dextran-coated iron oxide particles about 62-150 nm diameter, have been used clinically for liver diagnostics; up to 80% of these particles accumulate in the liver. The circulation half-life can be increased by using particles smaller than 50 nm. AMI25™ iron particles have also been tested for tumor imaging in bone marrow.

Protein based nanoparticles have been developed as high relaxivity contrast agents for molecular MRI [Merchant et al. IJRI 14(3). 2004; Uchida M, et al. Magn Reson Med 2008; 60(5):1073-1081; Bulte J W et al. JMRI. 4(3) 1994; Bennett K M, BioPhys Journal. 95(1) 2008]. With T1-weighting, agent concentrations of μM-mM are typically detected [Elleaume et al. Phys. Med. Biol. 2002 47:3369-3385]. However, in order to monitor the delivery of therapeutic agents, there is also significant interest in bimodal particle CT/MRI contrast agents [Caplan M R et al. ABME 33(8), 2005 Elleaume et al. Phys. Med. Biol. 2002 47:3369-3385-7; Regino et al. CM&MI 2008]. Dual CT/MRI agents have been reported to be used with concentrations greater than 47 mM of Gd [Regino et al. CM&MI 2008] but there remains a need for additional dual CT/MRI contrast agents.

### BRIEF SUMMARY OF THE INVENTION

The present invention provides methods and compositions for the production of a new tungsten-iron (W—Fe) ferritin nanoparticle, with a 4,497 mM-1s-1 and 458,143 mM-1s-1 per particle T1 and T2 relaxivities respectively, with visibility in CT at concentrations of 20 mM of tungsten (343 nM of particle). This nanoparticle can readily serve as a dual CT/MRI contrast agent.

### BRIEF DESCRIPTION OF SEVERAL VIEWS OF THE DRAWINGS

FIG. 1: TEM image of W—Fe nanoparticles (indicated by arrow) at a magnification of 110 k.

FIG. 2: Relaxivity values compared between Magnetoferritin and W—Fe Ferritin. Values are an average of 3 experiments.

FIG. 3: Relaxivity curves of W—Fe ferritin particles of (a) r1 and (b) r2.

FIG. 4: CT image of W—Fe ferritin compared to native ferritin (a) with intensity map of contrasted regions (b).

FIG. 5: In vivo T2 MRI image of rat striatum with injections of magnetoferritin and W—Fe ferritin as indicated.

FIG. 6: TEM images of (a) Native Ferritin, (b) W-magnetoferritin, (c) Magnetoferritin. Scale bars are 50 nm (d) HREM of W-magnetoferritin showing lattice fringes and multi-twinned crystal formation with lattice spacing of 2.5 A in each direction.

FIG. 7: EPR Spectrum of FeCl2 in dH2) showing no Fe(III) (bottom). Magnetoferritin showing characteristic peak at g=4.3 (middle), W-Magnetoferritin alloy decreased Fe (III) signal when compared to magnetoferritin (bottom).

### DETAILED DESCRIPTION OF THE INVENTION

The present invention relates to a new contrast agent that can be used for both CT scanning and MRI imaging. The contrast agent is a tungsten-iron (W—Fe) ferritin nanoparticle that has a visibility in CT at concentrations of 20 mM of tungsten (343 nM of particle). The nanoparticle has T1 and T2 relaxivities of 4,497 mM-1s-1 and 458,143 mM-1s-1 per particle respectively. This allows the nanoparticle to be used in an MRI imaging technique but also allows the same particle to allow acquisition of a CT image.

The term “contrast agent” is used herein to refer to a tungsten-iron-Ferritin molecule that generates a contrasting effect in vivo, whether the effect is direct or indirect or both.

The present invention uses ferritin as a component of the contrast agent. The term “Ferritin” is intended to include any of a group of diiron-carboxylate proteins characterized by the tendency to form a multimeric structure with bound iron and having a helix-bundle structure comprising an iron-coordinating Glu residue in a first helix and a Glu-X-X-His motif in a second. Certain ferritins maintain bound iron in a primarily Fe(III) state. Bacterioferritins tend to be haem proteins. Vertebrate ferritins tend to be assembled from two or more subunits, and mammalian ferritins are often assembled from a heavy chain and a light chain. Many ferritins form hollow structures with an iron-rich aggregate in the interior. Ferritin proteins are well known to those of skill in the art and some such proteins are described in further detail in U.S. Patent Publication 20060024662, which is incorporated herein by reference (see in particular sequences shown in the figures therein).

The compositions of the invention are used in performing various imaging of tissues and cells. For example, the invention contemplates methods of performing MRI using the tungsten-iron-ferritin contrast agents of the present invention. In such embodiments, the methods of the invention comprise contacting subject material with a composition comprising the contrast agent of the present invention and imaging the subject contacted using conventional CT scanning and/or MRI scanning. The contrast agents described herein may be employed in the imaging of essentially any biological material, including but not limited to: cultured cells, tissues, and living organisms ranging from unicellular organisms to multicellular organisms (e.g. humans, non-human mammals, other vertebrates, higher plants, insects, nematodes, fungi etc.). It is contemplated that the tungsten-iron-ferritin nanoparticles of the invention will be particularly useful in a combined CT scanning and MR angiography techniques.

In some aspects of the invention the contrast agents described herein may also contain one or more targeting moiety added to the composition. It should be noted that the targeting moiety may be added covalently bound to the tungsten-iron-Ferritin nanoparticle itself or may form part of the composition such as for example in a liposomal or other nanoparticle formulation. By “targeting moiety” herein is meant a functional group which serves to target or direct the tungsten-iron-Ferritin nanoparticle to a particular location, cell type, diseased tissue, or association. In general, the targeting moiety is directed against a target molecule and allows concentration of the compositions in a particular localization within a patient. Thus, for example, antibodies, cell surface receptor ligands and hormones, lipids, sugars and dextrans, alcohols, bile acids, fatty acids, amino acids, peptides and nucleic acids may all be attached to the tungsten-iron-Ferritin contrast agent of the invention to localize or target the nanoparticle compositions to a particular tissue site.

In another embodiment, the targeting moiety allows targeting of the nanoparticle compositions to a particular tissue or the surface of a cell.

In other embodiments, the targeting moiety is a peptide. For example, chemotactic peptides have been used to image tissue injury and inflammation, particularly by bacterial infection; see WO 97/14443, hereby expressly incorporated by reference in its entirety. Peptides may be attached via the chemical linkages to reactive groups on the exterior surface of the protein cage architectures (Flenniken, M. L., et al. 2005. Chemical Communications: 447-449), (Flenniken, M. L., et al. 2003. Nano Letters 3:1573-1576), (Gillitzer, E., et al. 2002. Chemical Communications: 2390-2391), (Hermanson, G. T. 1996. Academic Press, San Diego), (Wang, Q., et al. 2002. Chemistry & Biology 9:805-811; Wang, Q., et al. 2002. Chemistry & Biology 9:813-819; Wang, Q., et al. 2002. Angewandte Chemie-International Edition 41:459-462)). In some embodiments, peptides are attached to endogenous or engineered reactive functional groups on the exterior surface of each of the protein cage systems.

The peptides and other targeting moieties may be attached to the tungsten ferritin nanoparticle by use of chemical attachment. For example, activation of carboxylic acid groups and reaction with nucleophiles such as primary amines affords the coupling of ligands through formation of amide linkages. Engineered thiol functional groups (cys) on the protein may be modified by reaction with commercially available maleimide or iodoacetimide bifunctional linkers. In addition, synthetic methodologies developed for attachment through azide groups, and photochemical reactions of nucleophiles with tyrosine residues can be utilized. An exemplary technique for attachment includes click chemistry” (see Hartmuth, C. et al. (2001) Angewandte Chemie Int'l 40(11): 2004-21). Click chemistry is a modular protocol for organic synthesis that utilizes powerful, highly reliable and selective reactions for the rapid synthesis of compounds. For example, azides or alkynes are used as building blocks due to their ability to react with each other in a highly efficient and irreversible spring-loaded reaction.

In one embodiment, the attachment to a tungsten-iron-Ferritin nanoparticle of (i) proteins as targeting moieties and/or therapeutic agents and/or (ii) drugs as therapeutic agents, is achieved through the use of an azide linkage.

In one other embodiment, the attachment of proteins is achieved by a form of peptide ligation utilitzing an alkyne-azide cycloaddition reaction (Aucagne, V. et al. (2006) Sep. 28; 8(20): 4505-7).

In one aspect, the contrast agent compositions are used in a variety of imaging and therapeutic applications. For example, once synthesized, the contrast agent of the invention have use as magnetic resonance imaging contrast or enhancement agents. Specifically, the imaging agents of the invention have several important uses, including the non-invasive imaging of drug delivery, imaging the interaction of the drug with its physiological target, monitoring gene therapy, in vivo gene expression (antisense), transfection, changes in intracellular messengers as a result of drug delivery, etc.

Delivery agents comprising imaging agents comprising metal ions may be used in a similar manner to the known gadolinium MRI agents. See for example, Meyer et al., supra; U.S. Pat. No. 5,155,215; U.S. Pat. No. 5,087,440; Margerstadt et al., Magn. Reson. Med. 3:808 (1986); Runge et al., Radiology 166:835 (1988); and Bousquet et al., Radiology 166:693 (1988). The metal ion complexes are administered to a cell, tissue or patient as is known in the art.

A “patient” for the purposes of the present invention includes both humans and other animals and organisms, such as experimental animals. Thus the methods are applicable to both human therapy and veterinary applications. In addition, the contrast agents of the invention may be used to image tissues or cells; for example, see Aguayo et al., Nature 322:190 (1986).

Generally, sterile aqueous solutions of the imaging agent compositions of the invention are administered to a patient in a variety of ways, including orally, intrathecally and intraveneously in concentrations of from about 0.003 to about 1.0 molar, with dosages from about 0.03, about 0.05, about 0.1, about 0.2, and about 0.3 millimoles per kilogram of body weight being suitable. Dosages may depend on the structures to be imaged. Suitable dosage levels for similar complexes are outlined in U.S. Pat. Nos. 4,885,363 and 5,358,704.

Generally, the contrast agents of the invention will be formulated as pharmaceutical compositions for use with both imaging and therapeutic agents. The pharmaceutical compositions of the present invention comprise the contrast agent nanoparticles (which may optionally be loaded with therapeutic moieties) in a form suitable for administration to a patient.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

### Example 1

Synthesis of Nanoparticles:

The nanoparticles of the invention were synthesized from 2 μM native horse-spleen apoferritin (Sigma Aldrich, St. Louis), 48 mM Fe(II) Chloride (Sigma Aldrich, St. Louis), and 48 mM Sodium Tungstate Dihydrate (Sigma Aldrich, St. Louis) in 0.05M MES buffer (pH 8.5). The temperature of the apoferritin solution was monitored and maintained between 55 and 60° C. in a water bath and allowed to acclimate for 10 min prior to the initiation of synthesis. The solutions were continuously de-aerated with N2 gas (50 psi) the flow pressure was adjusted to reach a steady state until it gently bubbled thorough the solutions. Alternating 125 μL additions of Fe (II) Chloride and Sodium Tungstate Dihydrate were made at 5 minute intervals for a total of 20 additions of each compound. After approximately 10 additions the solution became rust in color and towards the end of synthesis the solution was yellow-brown. The solution was then dialyzed for 24 hours in a 10000 MW cut-off dialysis bag (BioDesignDialysis Tubing, Carmel, NY) against 3 L of 0.15 M Nail buffer. Using a 1.5T micro magnetic column (Miltenyi Biotech, Glad Bach, Germany), the dialyzed was magnetically filtered under a 0.15M NaCl buffer wash. The resulting protein concentration was obtained using the Better Bradford Assay (Thermo Scientific, Rockford). To confirm the presence of tungsten a sample of the solution was stained using 20% w/v Tin(II) Chloride (Sigma Aldrich, St. Louis) in 1.0M HCl. Upon confirmation, the concentrations of iron and tungsten were determined using inductively coupled plasma—optical emission spectroscopy.

Electron Microscopy:

Samples were imaged using a Philips CM12 electron microscope on Cu—C grids. Relaxometry: Several dilutions of sample suspended in 1% low-melt agarose gel were scanned in a 0.5T Bruker relaxometer. The Bruker's minispec software and exponential curve fitting feature were utilized to determine the T2 (Inter pulse τ=20 ms, 200 points) and T1 values (pulse separations ranging from 5 to 20000 ms, 4 scans, 7 points).

CT Imaging:

The sample was lyophilized and the concentrate imaged against native ferritin (Sigma Aldrich, St. Louis) using a Siemens AXIOM Sireskop SD System (60 kV, 2.5 mAs). ImageJ software was used to analyze the image.

In Vivo Imaging:

Using stereotactic injection, W—Fe contrast agent and magnetoferritin control were administered into the striatum of an adult male Sprague Dawley rat. To confirm the detectability in vivo, a 7T Bruker scanner and a surface RF coil was used, with a FLASH sequence (TE/TR=5.31/11.911 ms).

Results:

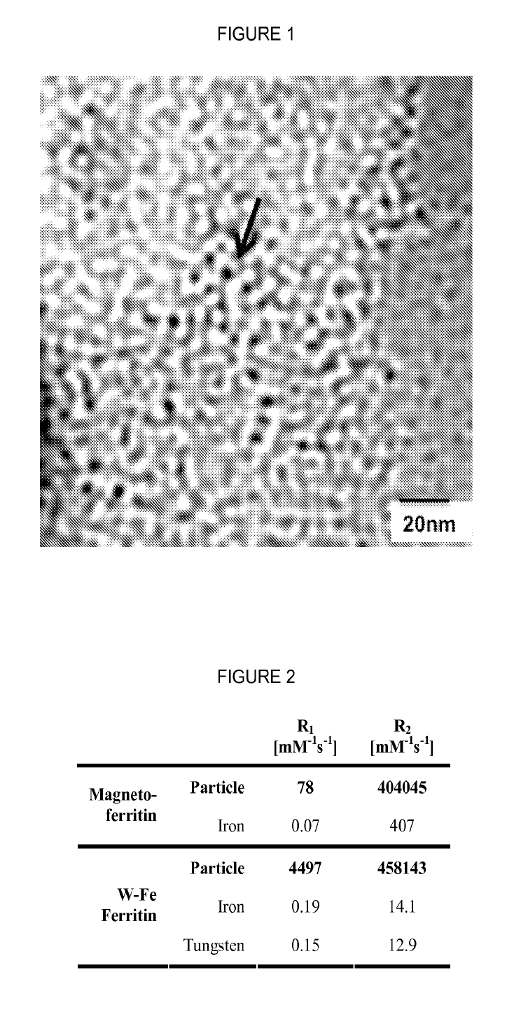

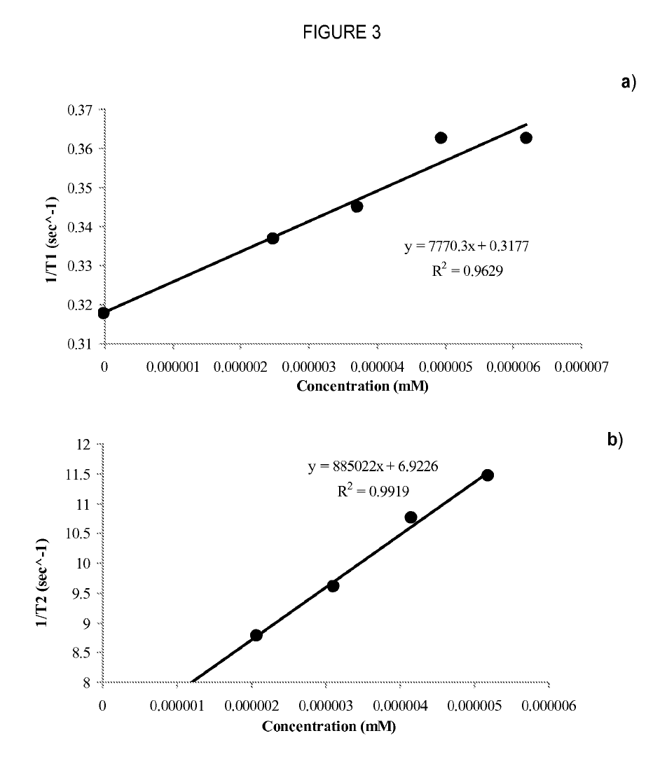

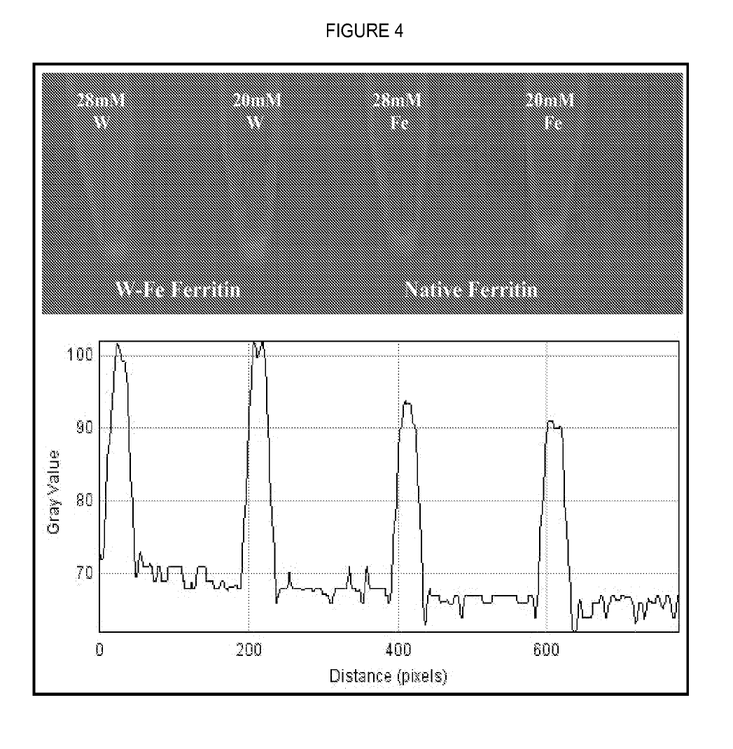

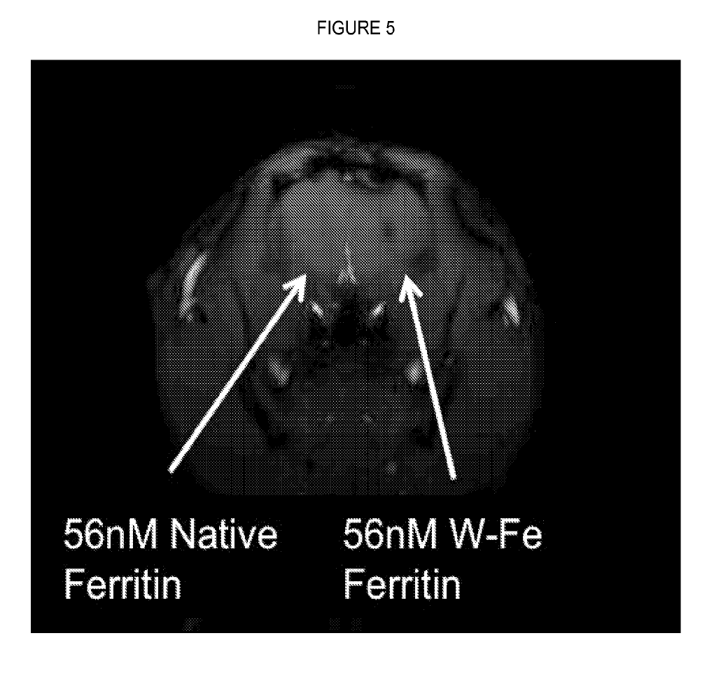

TEM visualization of the synthesized alloyed nanoparticles is shown in FIG. 1 and shows that the particles created range in size from 9 nm to 13 nm. Relaxivity measurements demonstrated a 58 fold increase in T1 relaxivity compared to magnetoferritin and a similar T2 relaxivity (FIG. 2), as obtained from the relaxivity curves (FIG. 3). Results of CT imaging demonstrated W—Fe ferritin contrast intensities on the order of 1.5 times greater than that of native ferritin for the same concentration, as shown in FIG. 4. In vivo results are shown in FIG. 5. W—Fe ferritin nanoparticles offer a high relaxivity, and show promise for dual modality CT/MRI applications.

### Example 2

The present example provides details of the use of the techniques of the present invention to increase the yield of magnetic nanoparticles. During synthesis, the addition of tungsten with iron oxide leads to a relaxivity (strength of the MRI contrast agent) that is approximately the same as the iron oxide, but leads to more particles in the solution being recovered. Thus, the present invention further comprises a method of using tungsten addition to increase the yield of magnetically filtered iron oxide contrast agents.

Ferritin has been used as a natural contrast agent, however, the protein in its native form possesses a weakly magnetic crystal core that as a relaxivity of ˜1-10 mM⁻¹s⁻¹. In order to increase per-ion and per particle relaxivity, one way of enhancing the magnetic properties for particles that are small enough to contain a single magnetic domain, less than ˜30 nm, is to create an alloy of different magnetic metals. In the present example, an alloy crystal is formed in the interior of the apoferritin cavity in an effort to enhance R₂ and increase the process yield. Although tungsten is diamagnetic, its inclusion in the crystal formed a tungsten-iron alloy with a per-particle relaxivity of 433,651 mM⁻¹s⁻¹ and per iron of 27666 mM⁻¹s⁻¹ and a percent yield increase of 200% compared to that of magnetoferritin.

Methods: Particle Synthesis:

A 2 μM apoferritin solution was buffered in 0.05M MES at pH 8.5, 48 mM FeCl₂ and 48 mM sodium tungstate dehydrate were de-aerated for 15 minutes with N₂. The solution was kept at a temperature of 55 to 60 degrees C. 125 μl of FeCl₂ was added to the apoferritin solution every 10 minutes for a total of 20 additions, after the 10^{th} addition 125 μl of sodium tungstate was added every 5 minutes after every FeCl₂ addition. Samples were dialyzed against 0.15M NaCl and filtered using a magnetic column and eluted into 0.15 NaCl buffer. As a protein control, 2 μM bovine serum albumin was used instead of aporferritin. Total protein concentration was obtained with a Bradford assay and inductively coupled plasma-optical emission spectroscopy (ICP-OES) was used to measure metal concentrations.

Relaxometry:

The particle relaxivity was measured using a 1.5T Bruker Minispec relaxometer. Bruekers curve-fitting tool was used to find the corresponding T₂ values (Inter-pulse μ=10 ms, 200 points) and T₁ values (pulse separations ranging from 5 to 20000 ms, 4 scans, 10 points) of samples suspended in a 1% agarose gel.

Electron Microscopy:

Particle samples were adsorbed on Cu—C grids and transmission electron microscopy (TEM) images were obtained using a Phillips CM12 electron microscope. High resolution electron microscopy (HREM) images were obtained using a Phillips CM200-FEG TEM/STEM.

Electron Spin Resonance:

EPR was performed with a X-band spectrometer with 5 mW power, 25G modulation and a temperature of 5K under liquid helium.

Results and Conclusions:

loading the apoferritin core with an alloy of tungsten and iron resulted in an increased in an increased per-iron and per-particle relaxivity (R₂) of 27,666 mM⁻¹s⁻¹ and 433,651 mM⁻¹s⁻¹ respectively (Table 1).

| | | Rₛ | R₂ |
|---|---|---|---|
| | | mM⁻¹ s⁻¹ | mM⁻¹ s⁻¹ |
| W- [symbol] | Fe | 80 | 27 [symbol] |
| | W | [symbol] | 2687 |
| | Particle | 1260 | 433 [symbol] |
| | [symbol] / | 1.93 | 4 [symbol] |
| | nm³ | | |
| [symbol] | Fe | 0.07 | 78 |
| particles | Particle | 407 | 4 [symbol] |
| | [symbol] / | 0.33 | [symbol] |
| | nm³ | | |

[symbol]

indicates data missing or illegible when filed

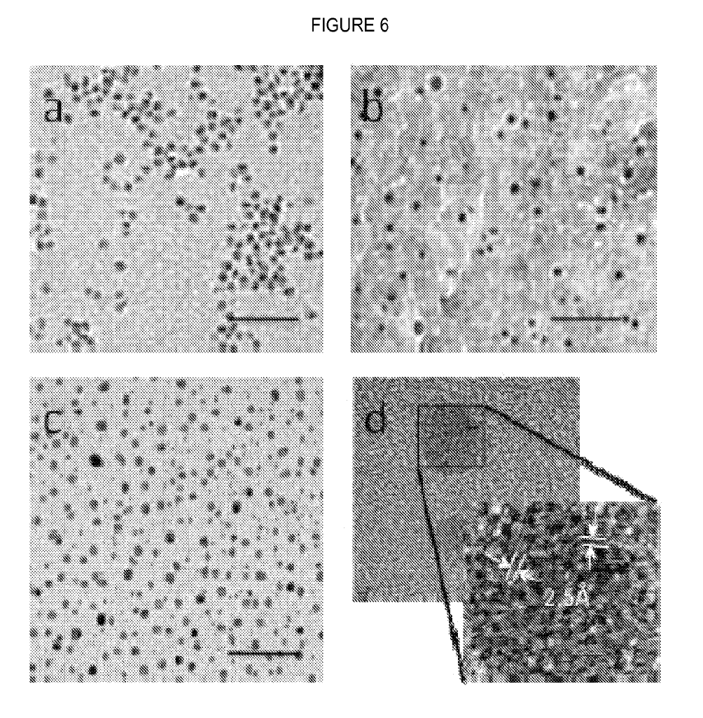

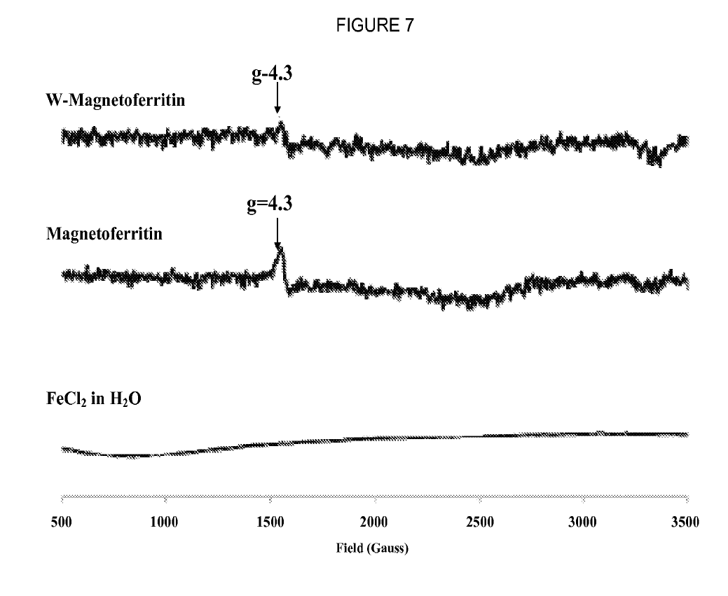

This synthesis procedure along with the addition of diamagnetic metal increased the nanoparticle yield after filtration by 200% when compared to magnetoferritin. Also ICP-OES indicated that ˜724 Fe ions and 7,454 tungsten ions are present within the protein. TEM showed the formation of electron dense metallic cores of mixed composition with diameters ranging from 5-7.5 nm which are larger than native ferritin and magnetoferritin (FIG. 6). HREM also showed that the crystal structures in the core are formed in a multi-twinned fashion each direction with lattice spacing of 2.5μ corresponding to magnetite (FIG. X3). Electron spin resonance showed that the newly synthesized W—Fe alloy nanoparticles had less Fe(III) in its cores compared to magnetoferritin. The presence of Fe(III) in the cores was confirmed by the typical iron peak at g=4.3. By contrast, FeCl₂ (a Fe(II) state) did not show paramagnetic signal in the spectrum (FIG. X3). This allows the conclusion that the magnetic properties (R₂) of magnetoferritin and the % yield can be strongly enhanced by addition of a diamagnetic metal into the synthesis to form an alloy crystal in the apoferritin cavity.

1. A contrast agent for imaging comprising a tungsten-iron (W—Fe)-ferritin nanoparticle wherein said contrast agent is both a CT imaging agent and a MRI imaging agent.

2. A contrast agent for imaging comprising: (a) an iron oxide nanoparticle, wherein the diameter of said iron oxide nanoparticle is between about 1 nm and about 500 nm; (b) a tungsten nanoparticle and (c) ferritin wherein said contrast agent has a higher relaxivity than the relaxivity of a contrast agent comprising native ferritin with iron oxide without tungsten.

3. The contrast agent of claim 1, wherein said contrast agent has a higher relaxivity than the relaxivity of a contrast agent comprising native ferritin without tungsten.

4. The contrast agent of claim 1, wherein said contrast agent has T1 and T2 relaxivities of 4,497 mM⁻¹S⁻¹ and 458,143 mM⁻¹S⁻¹ per particle, respectively.

5. The contrast agent of claim 1, wherein the visibility of said contrast agent in CT scanning at concentrations of 20 mM of tungsten.

6. The contrast agent of claim 1 wherein said contrast agent is present in nanoparticles of a size between particles range in size from 9 to 13 nm in diameter.

7. The contrast agent of claim 1, wherein said contrast agent has a contrast intensity that is greater than the contrast intensity of native ferritin of the same concentration.

8. The contrast agent of claim 6, wherein said contrast agent comprises at least a 25% increased CT image intensity as compared to the contrast intensity of native ferritin at the same concentration.

9. The contrast agent of claim 6, wherein the contrast intensity of said agent is 1.5 times greater than that of native ferritin for the same concentration.

10. The contrast agent of claim 1, wherein said nanoparticle further comprises a therapeutic agent and/or a targeting agent.

11. An improved ferritin-containing contrast agent wherein said contrast agent comprises a tungsten-iron (W—Fe) ferritin nanoparticle wherein presence of said tungsten in said contrast agent produces an increased contrast intensity of said contrast agent in CT imaging as compared to a ferritin containing contrast agent of the same concentration that does not contain tungsten.

12. An improved ferritin-containing contrast agent wherein said contrast agent comprises a tungsten-iron (W—Fe) ferritin nanoparticle wherein presence of said tungsten in said contrast agent produces an increased relaxivities of said contrast agent as compared to a ferritin containing contrast agent of the same concentration that does not contain tungsten in an amount effective to allow use of said contrast agent for both CT scanning and MRI scanning.

13. A composition comprising a contrast agent according to claim 1.

14. A method for in vivo imaging in a mammal of cells or tissues comprising the steps of: (a) administering to the mammal a composition of claim 13; (b) waiting a time sufficient to allow said composition to accumulate at a tissue or cell site to be imaged; and (c) imaging the cells or tissues with a non-invasive imaging technique whose resolution is enhanced by the presence of the dual modality contrast agent on or within the cells.

15. A method of claim 14, wherein in vivo image obtained from said method has a greater contrast than an image produced using ferritin in the absence of tungsten.
